(19) United States
(12) Patent Application Publication
(10) Pub. No.: US 2007/0203248 A1
(43) Pub. Date: Aug. 30, 2007

(54) Cardioprotective agents

(75) Inventors: Rama Mukherjee, Ghaziabad (IN); Anu T. Singh, Ghaziabad (IN); Kakali Dutta, Ghaziabad (IN); G.C. Maickap, Ghaziabad (IN); Anil Kumar Shukla, Ghaziabad (IN); Dhiraj Khattar, Ghaziabad (IN); Anand C. Burman, Ghaziabad (IN)

Correspondence Address: Ladas & Parry, 26 West 61st Street, New York, NY, 10023, US

(73) Assignee: DABUR RESEARCH FOUNDATION.

(21) Appl. No.: 11/789,974

(22) Filed: Apr. 26, 2007

Related U.S. Application Data
(63) Continuation of application No. 10/627,398, filed on Jul. 25, 2003.

(51) Int. Cl. A61K 31/137

(52) U.S. Cl. 514/649

(57) ABSTRACT

The invention relates to pharmaceutical compositions comprising 5-Methoxytryptamine or a salt thereof for the prevention and/or treatment of mammalian cardiac tissue damage. 5-Methoxytryptamine and the salts thereof act as free radical scavengers in the prevention and/or treatment of mammalian cardiac tissue damage mediated by free oxygen radicals.

### FIELD OF INVENTION

The invention relates to pharmaceutical compositions comprising 5-Methoxy tryptamine or a salt thereof for the prevention and/or treatment of mammalian cardiac tissue damage. 5-Methoxytryptamine and the salts thereof act as free radical scavengers in the prevention and/or treatment of mammalian cardiac tissue damage mediated by free oxygen radicals. More specifically the compositions containing 5-Methoxytryptamine or salt thereof can be used for the treatment of Doxorubicin induced cellular damage. The invention may also be extended to the treatment of other mammalian tissues viz. liver, kidney, intestine and brain.

### BACKGROUND OF INVENTION

The pineal gland secretes a number of pineal indoles including melatonin, methoxytryptophol, methoxytryptamine and other methoxyindoles and hydroxyindoles. The most extensively studied of the pineal indoles is melatonin (Burkhard, Poeggeler et al. J. Pineal. Res. 2002, 33: 20-30). The other pineal indoles have not been examined to the same depth. 5-Methoxytryptamine, one of the pineal indoles, is an agonist of 5-Hydroxy Tryptamine viz. Serotonin. It binds to the 5-HT₆ receptor subtype of serotonin, which may be exclusively localized to the central nervous system.

5-Methoxytryptamine is widely known to be an effective radioprotective agent (Kuna, P. et. al., Radiobiologia, Radiotherapia 1983, 24 (3), 365-76; Rozhdestvenskii, L. M. and Grozdov S. P. Radiobiologiya, 1979, 19(6), 868-75; Parzyck, D C. et al. Radiochemical and Padioanalytical Letters, 1974, 17(5-6), 351-358; Streffer, C. and Fluegel, M. Strahlentherapie, 1973, 146 (4), 444-449 and Feher, Imre et al., Int. J. Radiat. Biol, 1968, 14 (3), 257-262.)

5-Methoxytryptamine is also known to exert significant immunomodulating effects on cytokine secretion, consisting of inhibition of tumour necrosis factor alpha secretion with an anti-cachectic property (Sacco, S. et al. Eur. J. Pharmacol. 1998, 34: 249-255) and stimulation of IL2 and gamma interferon release with the following antitumour immunomodulatory effects (Sze, S. F. et al. J. Neural. Transm. Gen. Sect. 1993, 94, 115-126).

5-Methoxytryptamine has also been shown to possess free radical scavenging and anti-oxidative effects in hepatic and kidney tissues homogenates, mediated by a reduction in lipid peroxidation (Ng, T. B et. al, J. Neural Transmission 2000, 107(11), and this may be on account of its 5-methoxylic group (Chan, T. Y. and Tang, P. L., J. Pineal. Res. 1993, 14: 27-33).

Recently, pineal indoles like 5-Methoxytryptamine have been reported to possess oncostatic activity (Paolo Lissoni et al, Neuroendocrinol Letts, 2000: 21: 319-323).

Anthracycline antineoplastics are amongst the most active anticancer drugs and are effective against malignancies like leukemias, lymphomas and many solid cancers. These include Doxorubicin (sold under the trademark ADRIAMYCIN, NSC 123127, From Adria Laboratories, Columbus, Ohio), Daunorubicin, Epirubicin, THP-Adriamycin and Idarubicin. Doxorubicin is the drug of choice, alone or in combination with other chemotherapeutic agents, in the treatment of metastatic adenocarcinoma of the breast, carcinoma of the bladder, bronchogenic carcinoma, neuroblastoma, and metastatic thyroid carcinoma. It exerts its antitumour effects due to inhibition of DNA replication by intercalating between base pairs and/or steric inhibition of RNA activity.

Cardiotoxicity is the major limitation in the use of doxorubicin (Weiss, R. B., Semin. Oncol. 19, 670-686, 1992). The risk of developing cardiomyopathy becomes unacceptably high beyond the cumulative dose of 550 mg/m2 (Lefrak et al., Cancer 1973, 32, 302-314). In addition to clinical heart failure, cardiotoxicity encompasses clinical cardiotoxicity such as congestive heart failure and/or cardiac arrhythmias, and subclinical cardiotoxicity such as that detected by pathologic changes in cardiac biopsy or decrease in ventricular ejection fractions.

Thus, it has been found that doxorubicin treatment often must be terminated before the maximum effective cumulative dose has been administered to a patient bearing a neoplasm, because of the development of life-threatening cardiomyopathy. Thus, while doxorubicin is considered a highly effective anti-tumor agent, this effectiveness is significantly reduced by the concomitant cardiotoxicity encountered with use of the drug.

Doxorubicin induced cardiotoxicity is mediated through several different mechanisms including lipid peroxidation (Bordoni, A. et al., Biochim. Biophys. Acta 1999, 1440: 100-106), free radical formation (Yin, X. et. al., Biochem. Pharmacol., 1998, 56: 87-93, Hershko, C. et al, Leuk. Lymphoma 1993, 11: 207-214), mitochondrial damage (Cini Neri, G. et al., Oncology 1991, 48: 327-333), and iron dependent oxidative damage to biological macromolecules (Thomas, C. E. and Aust, S. D., Arch. Biochem. Biophys. 1986, 248: 684-689).

The complete mechanisms for doxorubicin and other anthracycline-induced cardiotoxicity are not completely understood. Three intracellular mechanisms are ascribed to Anthracyclines: interactions with DNA synthesis, binding to cell membranes and altering membrane functions, and intracellular Na⁺ & Ca²⁺ concentrations and stimulation of lipid peroxidation to form oxygen radicals (Young, R. C et. al, N. Engl. J. Med. 1981, 305: 139-153). It also may induce apoptosis in cardiomyocytes (Arola, O. J. et al., Cancer Res., 2000 Apr. 1, 60 (7): 1789-1792).

Further a pivotal role has been ascribed to iron in Doxorubicin induced cardiotoxicity (Minotti, G. et. al., FASEB Journal, 1999, 13: 199-212). Several studies indicate that anthracycline cardiotoxicity reflects disturbances in iron homeostasis within cardiomyocytes rather than the outcome of iron catalyzed reactions (Minnoti, G. et al., J. Clin Invest. 1995, 95: 1595-1605). Further the cardiotoxicity of Doxorubicin may be related to the inactivation of the iron regulatory protein by its metabolites (Minnnoti, G. et. al, FASEB, 1998, 12: 541-551).

The membrane interaction of Doxorubicin appears to be an integral part of the biochemical mechanisms of its toxicity. Chronic administration of Doxorubicin modulates the membrane bound adenylate cyclase and cAMP levels (Robison, T. W. and Giri, S. N., Virchows Arch. B cell Pathol. Incl. Mol. Pathol. 1987, 54(3): 182-189).

The effects of Doxorubicin on intracellular calcium homeostasis seems to be especially associated with the development of chronic cardiomyopathy (Young, R. C. et al., N. Engl. J. Med. 1981, 305: 139-153). Further Doxorubicin inhibits Na⁺/Ca²⁺ exchanger (Caroni, P., et al., FEBS Lett 1981, 130: 184-186), the oxygen consumption and the ATP production of mitochondria in in vitro rat heart preparation (Bachmann, E., et al., Agents Action 1975, 5: 383-393). Chronic dilated cardiomyopathy which can be induced by long term Doxorubicin treatment causes an upregulation of α and β adrenergic system as well as of the renin-angiotensin system (Kanda, T. et al., Eur. Heart J. 1994, 15, 686-690 and Morgan, H. E., Circulation, 1993, 87 IV4-IV6). Existing literature supports the view that one of the mechanisms may involve drug induced, cytotoxic, free radical formation (Buja et al., Cancer, 1973, 32, 771-778; Arena, E., et al., Int. Res. Commun. Syst. Med. Sci., 1974, 2, 1053-1061; Bristow, M. R. et al. Cardiovasc. Pharmacol., 1980, 2, 487-515). Further Doxorubicin administration is associated with a decrease in the presence of the endogenous antioxidants. Doxorubicin directly depresses cardiac glutathione peroxidase activity, the major defense against free-radical damage.

Pharmacological methods for development of novel cardioprotectives has involved the exploration of diverse classes of molecules.

At present, Dexrazoxane (ICRF-187, Zinecard), is an iron chelator, and is the only drug in human clinical use to reduce Doxorubicin induced cardiotoxicity (Swain, S. M. et al., J. Clin. Oncol., 1997: 15: 1333-1340, and Swain, S. M. et. al., J. Clin. Oncol., 1997, 15: 1318-1332).

Diverse classes of molecules or active principles of plants, have shown cardioprotective activities for Doxorubicin induced cardiotoxicity in animal models. These include lipid lowering drugs like Lovastatin (Feleszko, W. et al., Clin. Cancer. Res. Vol 6, 2044-2052, May 2000) and probucol (Li, T. and Singal, P., Circulation, 2000, 102: 2105-2110), cytoprotective drugs like Amifostine (Jahnukainen, K. et al., Cancer Research, 61, 6423-6427, Sep. 1, 2001), free radical scavengers like Vitamin E or N-acetylcysteine, calcium channel antagonists like Amlodipine (Yamanaka, S. et al., J. Am. Coll. Cardiol. 2003, Mar. 5, 41(5): 870-878), non selective β adrenoceptor blocker and vasodilator like Carvedilol (Santos, D. L. et al., Toxicol. Appl. Pharmacol., 2002 Dec. 15, 185(3): 218-227), Angiotensin converting Enzyme inhibitors like Captopril and Enalapril (El Aziz, M. A. et al., J. Appl. Toxicol., 21, 469-473, 2001) and plant extracts like curcumin (Venkatesan, N., Br. J. Pharmacol. 1998, June, 124 (3); 425-427).

Despite the Doxorubicin induced free radical formation (Yin, X. et al., Biochem Pharmacol, 1998,: 56: 87-93, Hershko, C. et al., Leuk. Lymphoma 1993, 11: 207-214) several of the well documented free radical scavengers and/or antioxidants do not protect against Doxorubicin induced cardiotoxicity in vivo. Studies with N-acetylcysteine or Vitamin E have shown that neither compound would prevent or significantly reduce cardiac lesions induced by chronic treatments with Doxorubicin (Herman, E. H. et al., Cancer Res., 1985, 45: 276-281 and van Vleet, J. F. et al., Am. J. Pathol, 1980, 99:13-22, Breed, J. G. et al., Cancer Research, Vol 40, No. 6, 2033-2038, 1980).

Similar negative results were obtained in clinical trials in which patients were given Vitamin E (Legha, S. S. et al., Ann. N.Y. Acad Sci 1982, 393:411-418) or N-acetylcysteine (Myers, C. et al., Semin. Oncol. 1983, 10 (suppl) 53-55) prior to and/or concomitant with Doxorubicin.

Inspite of the mixed results obtained with free radical scavengers they have been explored widely for their cardioprotective effects and also for protective effects in other tissues.

Reactive species induce several kinds of DNA damage, including single and double stranded DNA breaks, base and sugar modifications, DNA-protein crosslinks, depurination and depyrimidation, and alterations of biomembranes and circulating lipoproteins.

Free radicals are involved in myocardial reperfusion injury (McCord, J. M., Free Radic. Biol. Med. 1988, 4(1): 9-14; Downey, J. M., Ann. Rev. Physiol. 1990, 52: 487-505), and oxidative damage to the myocardium may represent a fundamental mechanism of myocardial injury (Loesser, K. E. et al., Cardioscience 1991, December, 2(4): 199-216). Free radical scavengers may have a role in reduction of myocardial ischemic injury (Gardner, T. J. et al., Surgery, 1983 September, 94(3), 423-427). There is a growing body of evidence suggesting a pathophysiological role of free radical mediated lipid peroxidation following central nervous system trauma or shock, which may be ischemic or hemorrhagic. Ischemia followed by reperfusion causes formation of oxygen-derived free radicals and increased lipid peroxidation and results in tissue injury. Species such as superoxide anions, hydroxyl, and peroxynitrite radicals are produced upon introduction of molecular oxygen into ischemic tissues (Ronson, R. S. et al., Cardiovasc. Res. 44(1): 47-59, 1999).

Administration of free radical scavengers to animals subjected to ischemia/reperfusion reduces these effects in heart, lung, kidney, pancreas, brain and other tissues. Further they may be useful in conditions viz. Atheroclerosis. Reactive oxygen species play a role in the formation of foam cells in atherosclerotic plaques (Steinberg, D. et al., New Engl. J. Med, 1989, 320: 915-924) and free radical scavengers like probucol have a marked antiatherosclerotic effect in hyperlipidemic animals (Carew, et al., Proc. Nat. Acad. Sci. USA, 1987, 84, 7725-7729). Further reactive oxygen species and their scavenging may have a role in treatment of intestinal ischemia (Kazez, A. et al., J. Pediatr. Surg. 2000 October, 35 (10): 1444-1448), and in renal ischemia (Dobashi, K., et al., Mol. Cell. Biochem. 2002, November, 240(1-2): 9-17, Sener, G. et al., J. Pineal Res. 2002, March, 32(2): 120-126) and in severe hepatotoxic responses (Wu J. and Zern M. A., Front Biosci. June, 1999, 15: 4: D520-D527).

Such compounds may be useful in the treatment of cancers, and degenerative diseases related to aging, stroke, head traumas (Halliwell, B. and Gutteridge, C. Biochem. J., 1984, 219, 1-14), and cataracts (Free Rad Biol Med: 12: 251-261, 1992), since oxygen derived free radicals have been identified among causative factors. There are several enzyme markers as well as circulating markers indicative of tissue damage (viz. creatine Kinase and Lactate Dehydrogenase) and those for primary cellular defense (viz. induction of Superoxide Dismutase and reduction in lipid peroxidation).

Inhibitors of brain lipid peroxidation counteract and reduce cerebral tissue damage (Hall E. D and Braughler, J. M., Free Radical Biology and Medicine, 1989, 6: 303-313, Miyamoto, M. et al., J. Pharmacol. Exp. Ther., 1989, 250, 1132).

Superoxide Dismutase is the most important enzyme involved in the primary cellular defense against reactive oxygen species such as hydrogen peroxide, superoxide anion and hydroxyl radicals generated in the cell. It decomposes the superoxide radicals to hydrogen peroxide which is in turn consumed by multiple enzymes such as catalase and glutathione peroxidase (Halliwell, B., Lancet 1994, 344: 721-724). Superoxide Dismutase is induced by hyperoxia (Crapo, J. D. and Tierney, D. F., Am. J. Physiol. 1974, 226: 1401-1407), irradiation (Oberley, L. W. et al., Arch. Biochem. Biophys., 1987,:54, 69-80) and changes in cellular redox status (Warner, B. B. et al., Am. J. Physiol., 1996: 271, L150-L158). Experiments have been conducted using Superoxide Dismutase therapy for the treatment of myocardial ischemia (Downey, J. M. et al., Free Radic. Res. Commun. 1991, 12-13 Pt2: 703-720).

Creatine kinase is an enzyme, which is readily measured in the blood of any individual with muscular tissue trauma or disease (Robinson, David J. et al., J. of Emergency Medicine, Vol 17, No. 1, pp 95-104, 1999). The cardiac specific isozyme of Creatine kinase CK-MB, further enhances the detection of myocardial infarction. Further CK-MB is produced exclusively in the myocardium, with very small amounts measured in the small intestine, tongue, diaphragm, uterus and prostate (Tsung, S., Clin. Chem. 1976, 22, 173). CK-MB measurements thus provide for a specific marker for identifying cardiac tissue damage and has become the “gold standard” for assessing myocardial infarction (Gillum, R. F. et al., Am Heart J, 1984, 108: 150-158). CK-MB is the only serum marker currently accepted in the World Health Organization (WHO) guidelines for the diagnosis of acute myocardial infarction (Gillum, R. F., et al., Am. Heart. J., 1984, 108: 150-158).

The enzyme Lactate dehydrogenase (LDH) catalyzes the reversible transfer of two electrons and hydrogen ion from lactate to NAD resulting in pyruvate and NADH. LDH is distributed in heart, kidney, brain, stomach and skeletal muscle. After an acute myocardial infarction (AMI), serum LD activities start to rise 12-18 hours after the onset of symptoms, and return to normal by 6-10 days (Wolf, P. L., Clin. Lab. Med. 1989, 9: 655). Elevated LDH levels are associated with a variety of pathological conditions.

In addition to the reported elevations in CK-MB and LDH levels in conditions such as AMI, acute administration of Doxorubicin is also reported to cause elevation in levels of both these enzymes (Saad, S. Y. et al., Pharmacol. Res., 2001, March, 43 (3): 211-218, El-Aziz, M.a. Abd et al., J. Appl Toxicol 21, 469-473, 2001, Mohamed, H. E., et al., Pharmacol. Res. 2000, August, 42 (2) 115-121).

5-Methoxy tryptamine (5-MT, Structure-I), and its salts, the subject of this invention, show promise as cardioprotectors for Doxorubicin induced cardiotoxicity in animal studies.

### SUMMARY OF THE INVENTION

The present invention is directed to pharmaceutical compositions of 5-Methoxy tryptamine or its salts useful in the prevention and/or treatment of mammalian cardiac tissue damage. More particularly, the invention provides a method for the prevention or treatment of mammalian cardiac tissue damage caused during Doxorubicin therapy. Also described are pharmaceutical compositions comprising 5-Methoxytryptamine or its salts for the prevention or treatment of damage to mammalian tissues including liver, kidneys, intestine, lung, pancreas and brain caused by free radicals.

Another aspect is the use of 5-Methoxy tryptamine or its salts for the prevention or treatment of damage to mammalian tissues including liver, kidneys, intestine, lung, pancreas and brain caused by free radicals.

### DETAILED DESCRIPTION OF THE INVENTION

5-Methoxytryptamine (available from M/s Aldrich) is represented by Structure I

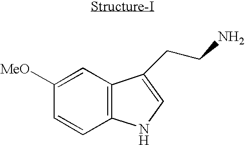

The present invention provides compositions and methods for presentation of 5-Methoxytryptamine and its salts in pharmaceutically acceptable form to patients undergoing doxorubicin treatment with a view to treat or prevent cardiotoxicity to myocardial tissue. In the compositions of this invention 5-Methoxytryptamine remains physically and chemically stable and can be administered in various dosage forms at the drug dose meant to be effective to exhibit clinically significant cardioprotective activity.

The present invention also provides compositions and methods for presentation of 5-Methoxytryptamine and its salts in pharmaceutically acceptable form to patients with a view to treat or prevent hepatotoxicity, nephrotoxicity and toxicity to other tissues like pancreas, intestine, lungs and brain caused by free radicals.

5-Methoxytryptamine can be used to prevent and/or treat cardiac toxicity, myocardial ischemia, myocardial infarction or heart failure.

5-Methoxytryptamine can be used in the prevention and/or treatment of certain diseases or conditions of the brain such as cerebral ischemia or cerebral infarction.

5-Methoxytryptamine can be used in the prevention and/or treatment of diseases or conditions of the coronary tissue or other blood vessels such as in the treatment or prevention of atherosclerosis or vascular injury following the reperfusion of obstructed arteries.

5-Methoxytryptamine can be used in the treatment of diseases or conditions of the kidney such as for the treatment of renal infarction or acute tubular necrosis.

5-Methoxytryptamine can be used in the treatment of diseases or conditions of the intestines such as for the treatment of intestinal ischemia or infarction.

Further the compounds of this invention may also be used for protection of hepatic, neural and renal tissues of animals/mammals treated with doxorubicin, adriamycin or other anthracycline antineoplastics.

The methods of this invention comprise, consist of, or consist essentially of administering orally, parenterally, or systemically to the mammal a therapeutically effective dose of 5-Methoxy tryptamine or its salts. An effective dose of 5-methoxy tryptamine or its salts thereof ranges from 0.7 to 7.0 mg/kg body weight, more preferably 1.2-5.0 mg/kg body weight, with the dose being dependent on the extent of effects sought and the manner of administration. This invention includes pharmaceutical compositions, containing 5-Methoxytryptamine or its pharmaceutically acceptable salts alongwith or in combination with one or more carriers, diluents, excipients and/or additives. The composition typically contains an amount of 5-Methoxytryptamine or a salt thereof effective to achieve the intended purpose. The unit dosage of a composition typically ranges from 5 mg-500 mg of 5-Methoxytryptamine or a salt thereof. An effective amount means that amount of a drug or pharmaceutical agent that will elicit the biological or medical response of a tissue, system, animal or human that is sought. In accordance with good clinical practice, it is preferred to administer the composition at a dose that will produce the effects sought without causing undue harmful side effects.

The term “salts” refers to salts prepared from pharmaceutically non-toxic bases including organic bases and inorganic bases. Representative salts include but are not limited to the following: acetate, ascorbate, benzoate, citrate, oxalate, stearate, trifluoroacetate, succinate, tartarate, lactate, fumarate, gluconate, glutamate, phosphate/diphosphate, and valerate. Other salts include Ca, Li, Mg, Na, and K salts, halides, salts of amino acids such as lysine or arginine; guanidine, ammonium, substituted ammonium salts or aluminium salts.

The salts of 5-Methoxy tryptamine may be prepared by methods known to those skilled in the art.

In one embodiment of the invention 5-Methoxytryptamine or its salts can be administered orally to human cancer patients by incorporating in a flavoured/sweetened syrup base.

5-Methoxytryptamine or its salts can be dissolved in a small amount of suitable solvent like water or alcohol. 5-Methoxytryptamine can be adsorbed onto inert excipients like colloidal silica to convert into a solid form that can be dispensed in a sachet, ampoule, vial, filled into hard gelatin capsules or into soft gelatin capsules. 5-Methoxytryptamine can be compressed into tablets with or without the addition of excipients. The formulations can be in the form of tablets, powders, capsules, lozenges, solutions, syrups, aqueous or oily suspensions, elixirs, implants, or aqueous or non-aqueous injections or any other forms that are pharmaceutically acceptable.

All the above delivery systems may contain added auxiliary agents such as fillers, diluents, preservatives, stabilizers etc.

The composition may be administered either alone or as a mixture with other therapeutic agents.

The in vitro and in vivo activity of the 5-Methoxytryptamine and its salts may be determined by standard assays that determine their free radical scavenging properties, effects on lipid peroxidation in cardiac homogenates, effects of the compounds on antioxidant enzymes viz. Superoxide Dismutase, Catalase, and on antioxidant peptides as on reduced Glutathione.

We have investigated the effect of 5-Methoxytryptamine on scavenging of free radicals in vitro, effect of 5-Methoxytryptamine on lipid peroxidation in live myocardial tissue, effect of 5-Methoxytryptamine on Superoxide Dismutase enzyme activity in live myocardial tissue, effect of 5-Methoxytryptamine on lipid peroxidation in live hepatic tissue, effect of 5-Methoxytryptamine on anticancer activity of Adriamycin in vitro; effect of 5-Methoxytryptamine on circulating levels of Creatine Kinase-MB (CK-MB) in Adriamycin treated animals, and effect of 5-Methoxy tryptamine on circulating levels of Lactate Dehydrogenase (LDH) enzyme in Adriamycin treated animals.

The present invention will now be illustrated by the following examples which are not intended to be limiting in any way.

### EXAMPLE 1

### Effect of 5 Methoxytryptamine on Scavenging of Free Radicals in Vitro

The free radical scavenging potential of 5-Methoxytryptamine was evaluated by the 1,1 diphenyl-2 picryl hydrazyl (DPPH) assay as described (Hycon, Lee et al, Arch. Pharm. Res. 19 (3), 223-227). Briefly 0.2 mM solution of 1,1 diphenyl-2 picryl hydrazyl was prepared in 100% methanol and immediately protected from light and kept at −20° C. 5-Methoxytryptamine was dissolved in 3.5% ethanol in normal saline and screened for its radical scavenging activity in concentration ranging from 1-1000 ug/ml. 100 ul of 0.2 mM DPPH was incubated with 100 ul of varying concentrations of 5-Methoxy tryptamine in 96 well tissue culture plates for 20 seconds at room temperature. All experiments were carried out in triplicates. 3.5% ethanol in normal saline was similarly incubated with 0.2 mM DPPH in control experiments for evaluating the effect of the vehicle on radical scavenging. The change in absorbance of DPPH incubated with varying concentrations of 5-Methoxy tryptamine of the vehicle was read at 517 nM for every 60 secs for 5 minutes. The absorbance of 0.2 mM DPPH taken after incubating for 5 minutes at room temperature was the blank O.D. The percent free radical scavenging ability of 5-Methoxy tryptamine was calculated as defined below.
Percent Free Radical scavenging=Blank OD at 5 minutes−Sample OD at 5 minutes×100/Blank OD at 5 minutes

Table 1 shows the percent radical scavenging ability of 5-Methoxytryptamine in vitro. As shown in Table 1, 5-Methoxytryptamine scavenges free radicals in a concentration ranging from 31-1000 ug/ml. It scavenges a maximal of 88.25% of free radicals at a concentration of 1000 ug/ml in vitro.

TABLE 1
Mean percent radical scavenging by 5-Methoxytryptamine in vitro.

| | Concentration of | Mean percent Radical |
|---|---|---|
| S. no | 5 Methoxytryptamine | scavenging |
| 1 | 31 ug/ml | 10 ± 4.3 |
| 2 | 62.5 ug/ml | 25 ± 5.0 |
| 3 | 125 ug/ml | 75.4 ± 5.5 |
| 4 | 250 ug/ml | 85.24 ± 4.8 |
| 5 | 500 ug/ml | 87.4 ± 5.0 |
| 6 | 1000 ug/ml | 88.25 ± 5.1 |

### EXAMPLE 2

### Effect of 5 Methoxytryptamine on Lipid Peroxidation in Live Myocardial Tissue

The effect of 5-Methoxytryptamine on lipid peroxidation in Adriamycin treated myocardial tissue was quantitated by Thiobarbituric acid reactive substances based assay as described (Uchiyama and Mihara, M., Anal. Biochem. 86, 271-278, 1978). Briefly male Wistar rats of the age group 5-6 weeks were maintained on normal rat pellets ad libitum. Rats were divided into four groups viz Groups I, II, III and IV.

Group I: Untreated

Group II, Animals treated with Adriamycin

Group III: Animals treated with 5-Methoxytryptamine and Adriamycin.

Group IV: Animals treated with 5 Methoxytryptamine

Each group consisted of 5 animals. 30 mg/kg body weight of Adriamycin was administered intraperitoneally to animals in Groups II and III. The animals comprising group III, were injected intraperitoneally with 5-Methoxytryptamine in concentration ranging from 8.5-35 mg/kg body weight 30 minutes prior to the Adriamycin treatment. The animals comprising group IV, were injected intraperitoneally with 5-Methoxytryptamine in concentration ranging from 8.5-35 mg/kg body weight. 24 hours later, the beating hearts of the animals were excised by decapitation. The heart tissue was washed in ice cold saline twice, weighed and snap frozen at −70° C. for assaying for Lipid peroxidation.

Briefly 200 mg of the live rat myocardial tissue was excised and homogenized in 2 ml of ice cold 10% Trichloro acetic acid buffer (TCA) buffer. To 200 ul of the homogenate thus obtained, 200 ul of 8.1% SDS, 1.5 ml of 20% Acetic acid, 1.5 ml of 0.8% of Thiobarbituric acid (TBA) and 1.0 ml of water was added in glass test tubes. The tubes were heated at 95° C. for 60 minutes. The mixture was cooled and diluted with 1 ml of double distilled water. A mixture of n-butanol and pyridine was prepared fresh in the ratio of 15:1 respectively. 5 ml of the n-butanol-pyridine mix was added to each tube containing the cardiac tissue homogenates. The tubes were centrifuged at 3000 rpm for 10 minutes at 4° C. 200 ul of the coloured liquid was collected from at the interphase of the aqueous and organic layers and the absorbance was measured spectrophotometrically at 532 nm. The control experiments contained only the TCA buffer treated similarly. The standard tubes contained Malonaldehyde in concentrations ranging from 2.5-25 uM. Malonaldehyde was dissolved in double distilled water. All experiments were carried out in triplicates. The extent of lipid peroxidation in the cardiac homogenates was expressed as uM/gm of the cardiac tissue. The extent of lipid peroxidation was calculated for the cardiac tissues of the animals comprising Groups I, II, III and IV. As shown in Table 2, 5-Methoxytryptamine in concentrations ranging from 8.5-35 mg/kg inhibits the lipid peroxidation in vivo in rat myocardial tissue treated with 30 mg/kg of doxorubicin. Further treatment with with 5 Methoxy tryptamine alone in concentrations ranging from 8.5-35 mg/kg did not alter the lipid peroxidation in vivo

TABLE 2
Lipid peroxidation (umoles/gm) in Adriamycin treated rat
myocardium in vivo

| | | Dose | | |
|---|---|---|---|---|
| Group | Treatment | Adriamycin | 5 MT | LP (umoles/g) |
| I | Untreated | Nil | Nil | 553 ± 10.5 |
| II | ADR | 30 mg/kg | Nil | 783 ± 21.0 |
| III | A (ADR & 5 MT) | 30 mg/kg | 8.5 mg/kg | 613 ± 20.1 |
| | B (ADR & 5 MT) | 30 mg/kg | 17 mg/kg | 625 ± 10.5 |
| | C (ADR & 5 MT) | 30 mg/kg | 35 mg/kg | 650 ± 11.2 |
| IV | A (5 MT) | Nil | 8.5 mg/kg | 535 ± 12.5 |
| | B (5 MT) | Nil | 17 mg/kg | 500 ± 14.5 |
| | C (5 MT) | Nil | 35 mg/kg | 513 ± 12.3 |

### EXAMPLE 3

### Effect of 5-methoxytryptamine on Superoxide Dismutase Enzyme Activity in Live Myocardial Tissue

The effect of 5-Methoxy tryptamine on Superoxide Dismutase activity in myocardial tissue was calculated as described (Kahhar et al., Indian Journal of Biochem. and Biophys. Vol. 21, April 1984, 130-132). Briefly male Wistar rats of the age group 5-6 weeks were maintained on normal rat pellets ad libitum. Rats were divided into four groups viz. Groups I, II, III and IV.

Group I: Untreated

Group II: Animals treated with Adriamycin

Group III: Animals treated with 5-Methoxytryptamine and Adriamycin.

Group IV: Animals treated with 5-Methoxytryptamine

Each group consisted of 5 animals. 30 mg/kg body weight of Adriamycin was administered intraperitoneally to animals in Groups II and III. The animals comprising group III, were injected intraperitoneally with 5-Methoxytryptamine in concentration ranging from 8.5-35 mg/kg body weight 30 minutes prior to the Adriamycin treatment. The animals comprising group IV, were injected intraperitoneally with 5 Methoxy tryptamine in concentration ranging from 8.5-35 mg/kg body weight. 24 hours later, the beating hearts of the animals were excised by decapitation. The heart tissue was washed in ice cold saline twice, weighed and frozen for assaying Superoxide Dismutase activity.

Briefly, 200 mg of rat myocardial tissue was excised and homogenized in 2 ml of ice cold Tris sucrose buffer (pH 7.4). The homogenate was centrifuged at 10000 rpm, at 4° C. for 10 minutes, and the supernatent carefully aspirated and collected. For each experiment, 1.2 ml of sodium pyrophosphate buffer was taken in clean glass tubes. To this 100 ul of 186 uM Phenazine methosulphate solution, 300 ul of a 300 uM solution of Nitroblue tetrazolium and 600 ul of double distilled water was added and mixed well. 600 ul of the supernatent obtained earlier was added per tube. A solution of NADH of the concentration 780 uM was freshly prepared for the experiments. The reaction in the tubes was initiated by the addition of 200 ul of a 780 uM solution of NADH per tube. The tubes were incubated for 90 seconds at room temperature. The reaction was stopped by the addition of 1 ml of 100% glacial acetic acid per tube and the absorbance measured spectrophotometrically at 560 nM. All experiments were carried out in triplicates. The control experiments contained only ice cold Tris Sucrose buffer (pH 7.4) treated identically. The standard tubes contained the enzyme Superoxide Dimutase dissolved in double distilled water in concentrations ranging from 0.1 U to 20 Units treated identically as above. The enzyme activity in the tissue homogenates was quantitated by rate of decrease in optical density at 560 nm, and expressed as units/mg protein. The Superoxide Dismutase activity was calculated for the cardiac tissues of the animals comprising Groups I, II, III and IV. As shown in Table 3, treatment with 5-Methoxytryptamine in concentrations ranging from 8.5-35 mg/kg increases the Superoxide Dismutase activity in vivo in rat myocardium treated with 30 mg/kg of Doxorubicin. Further treatment with 5-Methoxytryptamine alone in concentrations ranging from 8.5-35 mg/kg did not alter the Superoxide Dismutase activity in vivo.

TABLE 3
Superoxide Dismutase (U/mg) in Adriamycin treated
rat myocardium in vivo

| | | Dose | | |
|---|---|---|---|---|
| Group | Treatment | Adriamycin | 5 MT | SOD (U/mg) |
| I | Untreated | Nil | Nil | 35.1 ± 2.5 |
| II | ADR | 30 mg/kg | Nil | 3.3 ± 0.2 |
| III | A (ADR & 5 MT) | 30 mg/kg | 8.5 mg/kg | 5.95 ± 1.3 |
| | B (ADR & 5 MT) | 30 mg/kg | 17 mg/kg | 24.3 ± 2.5 |
| | C (ADR & 5 MT) | 30 mg/kg | 35 mg/kg | 28.1 ± 3.0 |
| IV | A (5 MT) | Nil | 8.5 mg/kg | 32 ± 2.5 |
| | B (5 MT) | Nil | 17 mg/kg | 30 ± 2.9 |
| | C (5 MT) | Nil | 35 mg/kg | 34 ± 4.5 |

### EXAMPLE 4

### Effect of 5 Methoxytryptamine on Lipid Peroxidation in Live Hepatic Tissue

The effect of 5 Methoxytryptamine on lipid peroxidation in Adriamycin treated hepatic tissue was quantitated by Thiobarbituric acid reactive substances based assay as described (Uchiyama and Mihara, M., Anal Biochem. 86, 271-278, 1978). Briefly male Wistar rats of the age group 5-6 weeks were maintained on normal rat pellets ad libitum. Rats were divided into four groups viz. Groups I, II, III and IV.

Group I: Untreated

Group II: Animals treated with Adriamycin

Group III: Animals treated with 5-Methoxytryptamine and Adriamycin.

Group IV: Animals treated with 5-Methoxytryptamine

Each group consisted of 5 animals. 30 mg/kg body weight of Adriamycin was administered intraperitoneally to animals in Groups II and III. The animals comprising group III, were injected intraperitoneally with 5-Methoxytryptamine in concentrations ranging from 8.5-35 mg/kg body weight 30 minutes prior to the Adriamycin treatment. The animals comprising group IV, were injected intraperitoneally with 5-Methoxytryptamine in concentrations ranging from 8.5-35 mg/kg body weight. 24 hours later, the liver tissue of the animals were excised. The liver tissue was washed in ice cold saline twice, weighed and snap frozen at −70° C. for assaying for Lipid peroxidation.

Briefly 200 mg of the rat liver tissue was excised and homogenized in 2 ml of ice cold 10% Trichloro acetic acid buffer (TCA) buffer. To 200 ul of the homogenate thus obtained, 200 ul of 8.1% SDS, 1.5 ml of 20% Acetic acid, 1.5 ml of 0.8% of Thiobarbituric acid (TBA) and 1.0 ml of water was added in glass test tubes. The tubes were heated at 95° C. for 60 minutes. The mixture was cooled and diluted with 1 ml of double distilled water. A mixture of n-butanol and pyridine was prepared fresh in the ratio of 15:1 respectively. 5 ml of the n-butanol-pyridine mix was added to each tube containing the liver tissue homogenates. The tubes were centrifuged at 3000 rpm for 10 minutes at 4° C. 200 ul of the colored liquid was collected from the interphase of the aqueous and organic layers and the absorbance was measured spectrophotometrically at 532 nm. The control experiments contained only the TCA buffer treated similarly. The standard tubes contained Malonaldehyde in concentrations ranging from 2.5-25 uM. Malonaldehyde was dissolved in double distilled water. All experiments were carried out in triplicates. The extent of lipid peroxidation in the liver homogenates was expressed as uM/gm of the liver tissue. The extent of lipid peroxidation was calculated for the liver tissues of the animals comprising Groups I, II, III and IV. As shown in Table 4, 5-Methoxytryptamine in concentrations ranging from 8.5-35 mg/kg inhibits the lipid peroxidation in vivo in rat liver tissues treated with 30 mg/kg Doxorubicin. Further treatment with 5 Methoxy tryptamine alone in concentrations ranging from 8.5-35 mg/kg does not alter the lipid peroxidation in vivo. Thus 5-Methoxytryptamine reduces the Adriamycin induced peroxidative damage to the liver tissue.

TABLE 4
Lipid peroxidation (umoles/gm) in Adriamycin treated
rat liver tissue in vivo

| | | Dose | | |
|---|---|---|---|---|
| Group | Treatment | Adriamycin | 5 MT | LP (umoles/g) |
| I | Untreated | Nil | Nil | 627 ± 11.5 |
| II | ADR | 30 mg/kg | Nil | 799 ± 11.0 |
| III | A (ADR & 5 MT) | 30 mg/kg | 8.5 mg/kg | 613 ± 210.1 |
| | B (ADR & 5 MT) | 30 mg/kg | 17 mg/kg | 625 ± 11.5 |
| | C (ADR & 5 MT) | 30 mg/kg | 35 mg/kg | 650 ± 15.2 |
| IV | A (5 MT) | Nil | 8.5 mg/kg | 601 ± 16.5 |
| | B (5 MT) | Nil | 17 mg/kg | 625 ± 15.5 |
| | C (5 MT) | Nil | 35 mg/kg | 630 ± 16.3 |

In view of the reported role of free radicals in causing tissue damage to diverse tissues as described earlier, the use of 5-Methoxytryptamine for the said purpose may be extended for the treatment for similar damage to other tissues viz, the brain, intestine, kidney, lung, and pancreas.

### EXAMPLE 5

### Effect of 5-Methoxytryptamine on Circulating Levels of Creatine Kinase-MB (CK-MB) in Adriamycin Treated Animals

The effect of 5-Methoxy tryptamine on circulating CK-MB levels was quantitated as described. Briefly male Wistar rats of the age group 5-6 weeks were maintained on normal rat pellets ad libidum. Rats were divided into four groups viz. Groups I, II, III and IV.

Group I: Untreated

Group II: Animals treated with Adriamycin

Group III: Animals treated with Adriamycin and 5-Methoxytryptamine

Group IV: Animals treated with 5-Methoxytryptamine.

Each group consisted of 6 animals. 5 mg/kg of Adriamycin was administered intraperitoneally to animals of groups II and III as two equal divided doses once every seven days. The animals comprising Group III were injected intraperitoneally with 17.5 mg/kg of 5-Methoxytryptamine, as seven equal doses given every alternate day. The animals of the group III were injected with 5-Methoxytryptamine, 30 minutes prior to the Adriamycin injection. The animals comprising Group IV were treated with 17.5 mg/kg of 5-Methoxytryptamine as seven equal doses, given every alternate day. At the 41^{st} day of the study, blood was collected from the retroorbital vein of all the animals in the study. The blood was kept at room temperature for 15 minutes, and spun at 3000 rpm for 15 minutes. The serum was separated and immediately estimated for the levels of CK-MB. The quantitation was carried out by kits procured from Bayer Diagnostics using RA 50 Chemistry Analyzer (Bayer Diagnostics), as per the manufacturers instructions.

As shown in Table 5, treatment with 5-Methoxytryptamine reduces the CK-MB levels in Adriamycin treated animals.

TABLE 5
CK - MB levels in Adriamycin treated animals.

| GROUP | CK - MB (U/L) |
|---|---|
| I (Untreated) | 382 ± 49 |
| II (Adriamycin treated) | 658 ± 19 |
| III (Adriamycin and 5- | 432 ± 38 |
| Methoxytryptamine treated) | |
| IV (5 Methoxytryptamine treated) | 379 ± 26 |

### EXAMPLE 6

### Effect of 5-Methoxytryptamine on Circulating Levels of Lactate Dehydrogenase (LDH) Enzyme in Adriamycin Treated Animals

The effect of 5-Methoxytryptamine on circulating LDH levels was quantitated as described. Briefly male Wistar rats of the age group 5-6 weeks were maintained on normal rat pellets ad libidum. Rats were divided into four groups viz. Groups I, II, III and IV.

Group I: Untreated

Group II: Animals treated with Adriamycin

Group III: Animals treated with Adriamycin and 5-Methoxytryptamine

Group IV: Animals treated with 5 Methoxytryptamine

Each group consisted of 6 animals. 5 mg/kg of Adriamycin was administered intraperitoneally to animals of groups II and III as two equal divided doses once every seven days. The animals comprising Group III were injected intraperitoneally with 17.5 mg/kg of 5-Methoxytryptamine, as seven equal doses given every alternate day. The animals of the group III were injected with 5-Methoxytryptamine, 30 minutes prior to the Adriamycin injection. The animals comprising Group IV were treated with 17.5 mg/kg of 5-Methoxytryptamine as seven equal doses, given every alternate day. At the 41^{st} day of the study, blood was collected from the retroorbital vein of all the animals in the study. The blood was kept at room temperature for 15 minutes, and spun at 3000 rpm for 15 minutes. The serum was separated and immediately estimated for the levels of LDH. The quantitation was carried out by kits procured from Bayer Diagnostics using RA 50 Chemistry Analyzer (Bayer Diagnostics), as per the manufacturer's instructions.

As shown in Table 6, treatment with 5-Methoxytryptamine reduces the LDH levels in Adriamycin treated animals.

TABLE 6
LDH levels in Adriamycin treated animals.

| GROUP | LDH (U/L) |
|---|---|
| I (Untreated) | 1018 ± 169 |
| II (Adriamycin treated) | 1296 ± 83 |
| III (Adriamycin & 5 Methoxy- | 1005 ± 38 |
| tryptamine treated) | |
| IV (5-Methoxytryptamine treated) | 1009 ± 26 |

### EXAMPLE 7

### Effect of Addition of 5-Methoxy tryptamine on Anticancer Activity of Adriamycin in vitro

Experiments were conducted to study the effect of addition of 5-Methoxytryptamine on the anticancer activities of Adriamycin in vitro in human tumour cell lines by performing the MTT cytotoxicity assay (Mosmann, T., J. Immunological Methods, 65:55; 1983). These cell lines included MiaPaCa2 (pancreatic cancer), DU145 (Prostate cancer), Breast (MCF7) and colon cancer (HT 29). Briefly, 10000 cells of the cultured human tumor cells were separately seeded per well in a 96-well culture plate and incubated with 5-Methoxytryptamine or Adriamycin or co-incubated with 5-Methoxy Tryptamine and Adriamycin. 5-Methoxytryptamine was dissolved in 3.5% ethanol in saline. Adriamycin was dissolved in saline immediately before use. The cells in the control experiments were treated with the appropriate concentrations of the vehicles The concentration of 5 Methoxytryptamine or Adriamycin varied from 1 ng/ml-1000 ng/ml. The effect of co-incubation of 5 Methoxytryptamine and Adriamycin on the cytotoxicity of Adriamycin were carried out at the ED₅₀ or ED₁₀₀ concentrations of Adriamycin co-incubated with 5 Methoxytryptamine at a concentration of 1 ug/ml. All experiments were carried out in triplicates at 37° C. in a CO₂ incubator. After 72 hours, the assay was terminated and percent cytoxicities and its ED₅₀ values calculated. Table 5 below shows that the ED₅₀ values of the cytotoxicity of Adriamycin is not altered by the addition of the 5-Methoxytryptamine at its highest concentration viz. 1 ug/ml.

TABLE 5
Effect of co-incubation of 5-Methoxytryptamine
and Adriamycin on the cytotoxicity of Adriamycin in vitro.

ED50 (Cytotoxicity ng/ml))

| | | | Adriamycin and 5- |
|---|---|---|---|
| S. No | Cell Line | Adriamycin | Methoxytryptamine |
| 1 | MCF 7 (Breast cancer) | 570 ± 4.5 | 571 ± 5.0 |
| 2 | DU145 (Prostate) | 98 ± 4.0 | 97 ± 4.0 |
| 3 | MiaPaca2 (Pancreatic) | 160 ± 5.0 | 160 ± 5.5 |
| 4 | HT29 (Colon) | 60 ± 5.0 | 60 ± 4.5 |

### EXAMPLE 6

### Preparation of Syrup of 5-Methoxytryptamine

1.0 gm of 5-Methoxytryptamine was dissolved in 5 ml of alcohol and added to 45 ml of sugar syrup already containing sufficient amounts of buffers, approved color, flavour and other stabilizers.

### EXAMPLE 7

### Preparation of Hard Gelatin Capsules of 5-Methoxytryptamine

According to the batch size required suitable amount of 5-Methoxytryptamine was mixed with excipients like lubricants and glidants exemplified by but not limited to talc, magnesium stearate, colloidal silica, etc. and filled into hard gelatin capsules.

### EXAMPLE-8

### Preparation of Injection of 5-Methoxytryptamine

100 mg of 5-Methoxytryptamine was dissolved in 0.3 ml of ethanol and made up the volume to one ml with surfactants exemplified by but not limited to polysorbates like polysorbate 80, polysorbate 60, polysorbate 20 etc, Cremophor ELP. Alternatively, the composition may contain cosolvents like PEG 300, Glycerol, Propylene glycol etc.

1. A composition comprising 5-methoxytryptamine or its a pharmaceutically acceptable salt and a carrier, excipient or additive; said 5-methoxytryptamine present in an amount effective to prevent, reduce or treat mammalian tissue damage.

2. The composition as claimed in claim 1 is a form selected from the group consisting of a tablet, capsule, powder, lozenge, solution, syrup, aqueous or oily suspension, elixir, implant, and aqueous or non-aqueous injection.

3. The composition as claimed in claim 1, wherein the amount of 5-methoxy tryptamine or its salt is from 5 to 500 mg.

4. (canceled)

5. (canceled)

6. (canceled)

7. A composition comprising 5-methoxytryptamine or its pharmaceutically acceptable salt and a carrier, excipient or additive.

8. The composition as claimed in claim 7 is a form selected from the group consisting of a tablet, capsule, powder, lozenge, solution, syrup, aqueous or oily suspension, elixir, implant, and aqueous or non-aqueous injection.

9. The composition as claimed in claim 7, wherein the amount of 5-Methoxy tryptamine or its salt is ranges from 5 to 500 mg.

10. A method for preventing tissue damage caused by exposure to an oxygen reactive species, comprising administering an amount of 5-Methoxytryptamine or a salt thereof effective to prevent tissue damage.

11. (canceled)

12. The method of claim 10, wherein the amount of 5-methoxytryptamine or its salt is 0.7 to 7.0 mg/kg body weight.

13. A method for treating tissue damage caused by exposure to an oxygen reactive species, comprising administering an amount of 5-Methoxytryptamine or a salt thereof effective to treat tissue damage to a patient in need thereof

14. (canceled)

15. The method of claim 13, wherein the amount of 5-Methoxytryptamine or its salt is 0.7 to 7.0 mg/kg body weight.

16. A method for treating cardiac toxicity, myocardial ischemia, myocardial infarction or heart failure comprising administering an effective amount of 5-Methoxytryptamine or a salt thereof to a patient in need thereof.

17. The method according to claim 16, wherein the cardiac toxicity is induced by an anthracycline antineoplastic.

18. The method of claim 16, wherein the amount of 5-methoxytryptamine or its salt is 0.7 to 7.0 mg/kg body weight.

19. A method for increasing the activity of superoxide dismutase enzyme in a tissue of a patient comprising administering to the patient an amount of 5-Methoxytryptamine or a salt thereof effective to increase the activity of superoxide dismutase enzyme.

20. (canceled)

21. A method for treating cardiac toxicity, myocardial ischemia, mycocardial infarction or heart failure comprising administration of an amount of 5-Methoxy tryptamine or a salt thereof effective to increase the activity of superoxide dismutase enzyme to a patient in need of such treatment.

22. A method for inhibiting lipid peroxidation in a tissue of a patient comprising administering to the patient an amount of 5-Methoxytryptamine or a salt thereof effective to inhibit the lipid peroxidation.

23. (canceled)

24. A method for treating cardiac toxicity, myocardial ischemia, mycocardial infarction or heart failure comprising administration of an amount of 5-Methoxytryptamine or a salt thereof effective to inhibit lipid peroxidation to a patient in need of such treatment.

25. A method for reducing levels of creatine kinase-MB in a tissue of a patient comprising administering to the patient an amount of 5-Methoxytryptamine or a salt thereof effective to reduce the level of creatine kinase-MB.

26. (canceled)

27. A method for reducing levels of lactate dehydrogenase in a tissue of a patient comprising administering to the patient an amount of 5-Methoxy tryptamine or a salt thereof effective to reduce the level of lactate dehydrogenase.

28. (canceled)

29. (canceled)

30. (canceled)

31. (canceled)

32. The method according to claim 27, wherein the tissue is myocardium, liver, kidney, intestine, pancreas or brain.

33. The method according to claim 10, wherein the 5-methoxytryptamine or the pharmaceutically acceptable salt thereof is administered in a composition comprising 5-methoxytryptamine or the pharmaceutically acceptable salt and a carrier, excipient or additive.

34. The method according to claim 33, wherein the composition is in a form selected from the group consisting of a tablet, capsule, powder, lozenge, solution, syrup, aqueous or oily suspension, elixir, implant, and aqueous or non-aqueous injection.

35. The method according to claim 33, wherein the amount of 5-methoxy tryptamine or the salt in the composition is from 5 to 500 mg.

36. The method according to claim 13, wherein the 5-methoxytryptamine or the pharmaceutically acceptable salt is administered in a composition comprising 5-methoxytryptamine or the pharmaceutically acceptable salt and a carrier, excipient or additive.

37. The method according to claim 36, wherein the composition is in a form selected from the group consisting of a tablet, capsule, powder, lozenge, solution, syrup, aqueous or oily suspension, elixir, implant, and aqueous or non-aqueous injection.

38. The method according to claim 36, wherein the amount of 5-methoxy tryptamine or the salt in the composition is from 5 to 500 mg.

39. The method according to claim 16, wherein the 5-methoxytryptamine or its pharmaceutically acceptable salt is administered in a composition comprising 5-methoxytryptamine or the pharmaceutically acceptable salt and a carrier, excipient or additive.

40. The method according to claim 39, wherein the composition is in a form selected from the group consisting of a tablet, capsule, powder, lozenge, solution, syrup, aqueous or oily suspension, elixir, implant, and aqueous or non-aqueous injection.

41. The method according to claim 39, wherein the amount of 5-methoxy tryptamine or the salt in the composition is from 5 to 500 mg.

42. The method according to claim 16, wherein the cardiac toxicity, myocardial ischemia, myocardial infarction or heart failure is treated by reducing levels of creatine kinase-MB in the cardiac tissue of the patient by administering to the patient an amount of 5-Methoxytryptamine or the salt thereof effective to reduce the level of creatine kinase-MB.
